(19) United States
(12) Patent Application Publication
(10) Pub. No.: US 2015/0038940 A1
(43) Pub. Date: Feb. 5, 2015

(54) APPLIANCE FOR PERFORMING ANAESTHESIA OR ANALGOSEDATION, AND METHOD FOR OPERATING AN APPLIANCE FOR PERFORMING ANAESTHESIA OR ANALGOSEDATION

(71) Applicants: Universitaet des Saarlandes, Saarbruecken (DE); KIST Europe Forschungsgesellschaft mbH, Saarbruecken (DE)

(72) Inventors: Sascha Kreuer, Zweibruecken (DE); Thomas Volk, Glienicke-Nordbahn (DE); Heiko Buchinger, Saarbruecken (DE); Joerg-Ingo Baumbach, Dortmund (DE)

(21) Appl. No.: 14/384,320

(22) PCT Filed: Mar. 13, 2013
(86) PCT No.: PCT/DE2013/200006

§ 371 Date: Sep. 10, 2014

(30) Foreign Application Priority Data
Mar. 13, 2012 (DE) 10 2012 203 897.3

(51) Int. Cl. A61M 5/172; A61M 5/145

(52) U.S. Cl. CPC A61M 5/1723; A61M 5/1452; A61M 2230/005; A61M 2230/437; A61M 2230/432; A61M 2230/10; A61M 2205/3306; A61M 2202/048; A61M 2205/50
USPC 604/503; 604/66

(57) ABSTRACT

The invention relates to an appliance for performing anaesthesia or analgosedation, comprising a dosing device (5) for the intravenous administration of an adjustable dose of at least one anaesthetic to a patient (1), a measuring device (2) for determining the concentration of the at least one anaesthetic in the exhaled air of the patient (1), means (4) for determining the effect of the at least one anaesthetic on the patient (1), preferably in the form of a depth of anaesthesia or depth of analgosedation, and a data processing device (7), which communicates via interfaces with the dosing device (5), the measuring device (2) and the means (4) for determining the effect and which, on the basis of the determined values of the parameters dosage, concentration and effect of the at least one anaesthetic, establishes a pharmacological model individualized to the patient (1) and, using the individualized pharmacological model that is established, calculates an individual dosage, optimized to the patient (1), of the at least one anaesthetic.

### CROSS REFERENCE TO RELATED APPLICATIONS

This application is a national stage application, filed under 35 U.S.C. §371, of International Application No. PCT/DE2013/200006, filed Mar. 13, 2013, which claims priority to German Application No. 10 2012 203 897.3, filed Mar. 13, 2012, the contents of both of which as are hereby incorporated by reference in their entirety.

### BACKGROUND

1. Technical Field

The invention relates to an apparatus for the performance of anesthesia or analgo-sedation, as well as a method for operating an apparatus for the performance of anesthesia or analgo-sedation.

2. Description of Related Art

Under general anesthesia or narcosis, certain body functions are switched off for the purpose of tolerance of diagnostic or operating interventions on or in the body. In general, the aim of adequate anesthesia is to obtain a combined effect of hypnotic, analgesic and muscle relaxing effects, thus ensuring that the patient is in a state of unconsciousness during the intervention and does not perceive the intervention, and that he is also insensitive to pain stimuli during surgery.

In the case of analgo-sedation, a stepped form of the depth of anesthesia is desired that offers a combination of hypnotic and analgesic effects without the effect of muscle relaxants.

To achieve the above objectives, the anesthesiologist usually administers a combination of anesthetic drugs having different effects on the brain, the spinal cord, the autonomic nervous system and/or neuromuscular junctions. As a rule, for example, narcotics/sedatives for unconsciousness, sedation or tranquilization are combined with analgesics used for pain suppression. A commonly-used drug from the group of anesthetics is propofol (active ingredient: 2,6-diisopropylphenol), while opioids, such as remifentanil, fentanyl or morphine, are typically used as analgesics.

For the anesthesiologist, not only the actual selection of appropriate drugs represents a major difficulty, but also the dosing of these appropriately. In this context, it is vital to avoid overdoses as far as possible, as these may lead to unwanted side effects with potentially serious consequences. In addition, an overdose would unnecessarily increase the overall burden on the patient and excessively prolong anesthesia. On the other hand, the doses must not be too low because, for example, this could lead to insufficiently deep anesthesia with the patient consciously experiencing the intervention, which might lead to serious resultant traumas. An adequate dosage must be guaranteed for the entire duration of diagnostic or invasive surgery.

The problem of determining an adequate dosage is difficult because only limited information is available to the anesthesiologist on the concentration of the administered anesthetic at the site of action. For many years in the case of gaseous anesthetics, it had been known how to determine the concentration at the end of the expiration of the patient—end-tidal. This measurement, which provides a relatively reliable indicator of the anesthetic effect, is mandatory and serves the anesthetist for metering the supply of the anesthetic. In the case of (non-volatile) intravenously administered anesthetics, however, there was no way of measuring the concentration.

For example, the dosage of propofol may be determined using a computer-aided syringe pump (Target Controlled Infusion, TCI), which infuses the drug on the basis of pharmacokinetic data. The correlation between the propofol concentration in the patient's blood and the administered dose is calculated solely on the basis of the demographic data of the patient, such as height, weight, age, gender. The pharmacological models that are stored in the TCI syringe pump, as found today in clinical practice, have an accuracy of about 20% in the case of healthy patients. In patients with organ dysfunction, there is an even larger deviation. Other limitations exist, for example in obese patients and in children. Accordingly, and based on these models, anesthesia control is necessarily imprecise.

### BRIEF SUMMARY

The present invention, therefore, has the object of designing and developing an apparatus for performing anesthesia or analgo-sedation, as well as a method for operating an apparatus for performing anesthesia or analgo-sedation, and, further, to make possible anesthesia control with improved accuracy.

According to the invention, the foregoing object is achieved by the features recited in the claims. In this case, the device for the performance of anesthesia or analgo-sedation comprises a metering device for intravenous administration of an adjustable dose of at least one anesthetic agent to a patient, a measuring device for determining the concentration of at least one anesthetic agent in the air exhaled by the patient, means to determine the effect of the at least one anesthetic agent in the patient, preferably in the form of anesthetic or analgo-sedation depth, and a data processing device that communicates via interfaces with the metering device, the measuring device and the means to determine the effect, in order to produce a pharmacological model that is individualized for the patient on the basis of the determined values of the parameters for the dosage, concentration and effect of the at least one anesthetic agent, to thus calculate a dosage of the at least one anesthetic agent that is optimally customized for the patient on the basis of the individualized pharmacological model.

In procedural terms, the foregoing object is also achieved by the features recited in the claims. Hereinafter, the method for operating a device for performing anesthesia comprises the steps:
Intravenous administration of an adjustable dose of at least one anesthetic agent to a patient;
Determination of the concentration of the at least one anesthetic agent in the exhaled air of the patient;
Determination of the effect of the at least one anesthetic agent on the patient, preferably in the form of anesthetic depth,
Creation of a customized pharmacological model for the patient based on the parameters representing the dosage, concentration, and effect of the at least one anesthetic agent or, respectively, determined values, and
Determination of a dosage of the at least one anesthetic agent that is optimized for the patient on the basis of the individualized pharmacological model.

In accordance with the invention, it has been firstly recognized that improved accuracy with respect to the performance of anesthesia or analgo-sedation may be achieved and integrated during surgery on patients based on real-time or quasi real-time data obtained in a pharmacological model. According to the invention, a measuring device to determine the concentration of anesthetic agent in the exhaled air of the patient, means for determining the effect (anesthetic or analgo-sedation depth) of the administered anesthetic agent, as well as a metering device for the intravenous administration of an anesthetic agent via a data processing device, are networked with each other. The measured concentration values flow together with information regarding the effect into an individual pharmacological model that is tailored to the individual patient. It is preferable that the pharmacological model represents a complete PK/PD model that takes into account both the pharmacokinetic and pharmacodynamic aspects. Patient-specific anesthetic or analgo-sedation control may be effected in parallel with the calculation of such an individually customized pharmacological model for each patient during the intervention on the patient.

With respect to as an exact dosage of the anesthetic agent as possible and in the context of a specific embodiment, the dispensing system includes a computer-controlled syringe pump. This enables the anesthesiologist to determine simple and accurate replenishment of anesthetic agent as needed during surgery. The syringe pump is characterized by its continuous delivery of the administered dosage of the respective anesthetic agent to the patient during surgery and transmission of the data via a corresponding interface to the data processing device.

In a preferred embodiment, the measuring device works continuously in the determination of the concentration of the at least one anesthetic agent, whereby the resulting discontinuous respiratory gas flow is transferred to a continuous sampling gas flow, while the latter is supplied to a sensor system of the measuring device. As an alternative to a continuous examination of the exhaled air and the corresponding determinations of the concentration, the measurements may also be made at short measurement intervals of less than 60 s, ideally less than 30 s, and preferably in a range of 15-25 s, so that a current concentration value that may be included in the PK/PD model is provided about every 3-5 breaths of the patient. In order to obtain the above-mentioned short measuring intervals, the measuring device is advantageously in the form of an ion mobility spectrometer with upstream gas-chromatographic separation columns, preferably multi-capillary columns. The separation columns/multi-capillary columns allow a preliminary separation of the individual components present in the breathing gas, so that the individual components occur at different times in the drift tube of the ion mobility spectrometer and/or have different drift times/mobilities. Accordingly, it is possible to determine the concentration of several different anesthetic agents independently, and virtually in parallel with one another.

The defined removal of the exhaled gas samples, both in terms of their respective volumes as well as in terms of the respective breathing phases, is of vital importance for the validity of the concentration measurements of the exhaled air of a patient. Advantageously, therefore, the ion mobility spectrometer is coupled with a volume flow sensor (flow sensor) and/or with a CO₂ sensor. In this way, it is possible to supply uniform breathing gas volumes depending on a defined content of CO₂, through which a specific breathing phase (for example, expiration, end-tidal, etc.) is defined, and supplied to the sensor system. The concentration may be determined through the determination of the volume of such a dosing loop, preferably between 1 ml and 50 ml.

In a preferred embodiment, the means for the determination of the effect of the at least one anesthetic agent on the patient comprises a device for deriving an EEG, hereinafter referred to as an EEG module.

In a preferred embodiment and in addition to the values of the dosage, concentration and effect parameters, demographic data of the patient is integrated into the individualized PK/PD model to achieve a more far-reaching individualization. The demographic data of the patient, especially age, weight, height, gender and BMI (Body Mass Index) may be either manually entered using the corresponding entry means of the data processing device or read directly from a patient database into the data processing device.

The at least one anesthetic agent for which, taking into account the measured concentration values in the air exhaled by the patient, an individualized patient-specific PK/PD model is created, may, for example, be a narcotic, especially propofol. Additionally or alternatively, the at least one anesthetic agent may comprise an analgesic, in particular an opioid. Interaction models may be generated, for example, between propofol and an opioid on the basis of measured concentration values and their integration into a PK/PD model. This is of particular advantage when, apart from propofol, a hypnotic opioid is used as the analgesic in the vast majority of operational interventions. By using such interaction models, one takes into account the fact that most analgesics, particularly the most common opioids, have a component with a hypnotizing effect in addition to the component with an analgesic effect. It should be noted at this point that for the production of the interaction models, the time intervals for the determination of the concentration values of the narcotics and opioids need not necessarily be identical, but may in fact differ from one another.

Moreover, it is also possible that the at least one anesthetic agent comprises a muscle relaxant in order to take into account the measured concentration values in the exhalation air of the patient in the creation of the individualized patient-specific PK/PD model.

In the context of a specific embodiment, it is provided that the apparatus is designed in the sense of an “open loop” system. For this purpose, for example, a display device may be provided, on which the dose of the anesthetic agent(s), as optimally calculated for the patient, is represented as a recommendation. The anesthetist may then decide, taking into account the current overall anesthesia situation, whether the recommendation should be followed and the dosage adjusted accordingly.

Alternatively, the apparatus may be designed in the form of a “closed loop” system. In this variant, the patient-specific optimal dosage of the anesthetic agent is used to generate appropriate control signals calculated on the basis of the individualized PK/PD model, which is transmitted to the metering device for the automatic adjustment of the dosage.

In an advantageous embodiment, the data processing device is arranged to perform a correlation analysis between the EEG index values determined by the EEG module and the measured concentration of the at least one anesthetic agent in the exhaled air of the patient.

### BRIEF DESCRIPTION OF THE FIGURES

There are various ways of advantageously designing and further developing the teaching of the present invention. On the one hand, there are the respective dependent form patent claims, while, on the other hand, there is the following description of preferred exemplary embodiments of the invention with reference to the drawing. In connection with the discussion of preferred exemplary embodiments of the invention with reference to the drawing, preferred embodiments and further developments of the teaching will also be explained in general. In the drawing,

FIG. 1 shows a schematic representation of an exemplary embodiment of a device for performing anesthesia according to the invention, and

FIG. 2 shows a schematic representation of a method to create an individual patient-specific PK/PD model according to an exemplary embodiment of the invention.

### DETAILED DESCRIPTION OF VARIOUS EMBODIMENTS

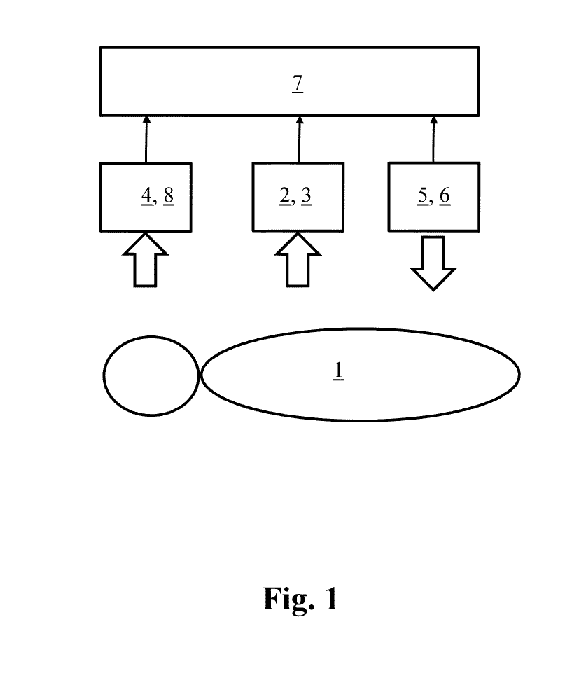

FIG. 1 shows a schematic representation of a preferred exemplary embodiment of a device according to the invention for performing anesthesia, which could be directly transferred to the performance of analgo-sedation. The patient 1 and the essential components of the device are shown. Specifically, the illustrated device comprises a measuring device 2 to determine the concentration of an anesthetic agent in the exhaled air of the patient 1 in the form of an ion mobility spectrometer (IMS) 3 with multi-capillary columns (MCC), an EEG module 8, a metering device 5 for intravenous administration of an adjustable dose of an anesthetic agent to a patient in the form of a TCI syringe pump 6, as well as a data processing device 7. The functionality of these modules will be described in more detail below, whereby, as an example, it is assumed that the intravenously administered drug in the context of the described anesthesia is propofol, because propofol is today the most widely used anesthetic for general anesthesia and sedation. However, the following remarks may be applied to other administered anesthesia drugs. In particular, the following embodiments may be applied to a number of different drugs that are administered in parallel to the patient during the intervention, whereby interaction models for the individual drugs may be produced in such cases, for example to describe the interaction of propofol with an opioid and/or a muscle relaxant drug.

The IMS 2 measures the current propofol concentration in the air exhaled by the patient 1 continuously or at regular intervals. In the case of temporally offset measurements, these are performed with a maximum time interval of about 30 s. These short measurement intervals ensure that the measurements are quasi real-time measurements. The measured values are accordingly immediately available online during the intervention.

To measure the concentration of propofol, in addition to the IMS 2, a breathing gas sensor (not shown) is provided that is particularly in the form of a CO₂ sensor or a flow sensor, and the CO₂ concentration is measured in the exhalation phase. The respiratory gas sensor serves to control the removal of the sample gas from the respiratory gas flow. As soon as the breathing gas sensor detects a concentration of CO₂ in the exhalation phase that exceeds a first predetermined value, the sample gas removal begins. Once the CO₂ concentration falls below a second predetermined value, the sample gas removal is terminated. In this way, reproducible samples are obtained that are always from the same defined breathing phase. The samples so obtained are then fed to the IMS 2 in order to determine the exact concentration of propofol.

In parallel with the measurement of the concentration of propofol, an EEG of the patient 1 is derived by means of the EEG module 4. The EEG is displayed on a corresponding EEG monitor for the anesthesiologist. Moreover, index values, for example so-called BIS values (Bispectral Index Monitoring), are transmitted from the EEG monitor and likewise displayed. These EEG index values are dimensionless and are usually defined on a scale of 0 to 100 and represent a measure of the depth of hypnosis.

As shown in FIG. 1, the EEG values, the measured propofol concentrations as well as the dose of propofol administered to the patient 1 are transmitted to a data processing device 7 via corresponding interfaces. According to the invention, an individualized pharmacological model—a PK/PD model—is created for the patient 1 based on these values. Based on this PK/PD model, an optimized individual propofol dosage is then calculated for each patient 1. The propofol dosage optimized in this way may then be made available to the anesthesiologist through a corresponding output or display means in the form of a recommendation.

Alternatively, a control loop may be established, whereby in this case the optimized propofol dose is immediately transmitted as a corresponding control signal to the syringe pump 6.

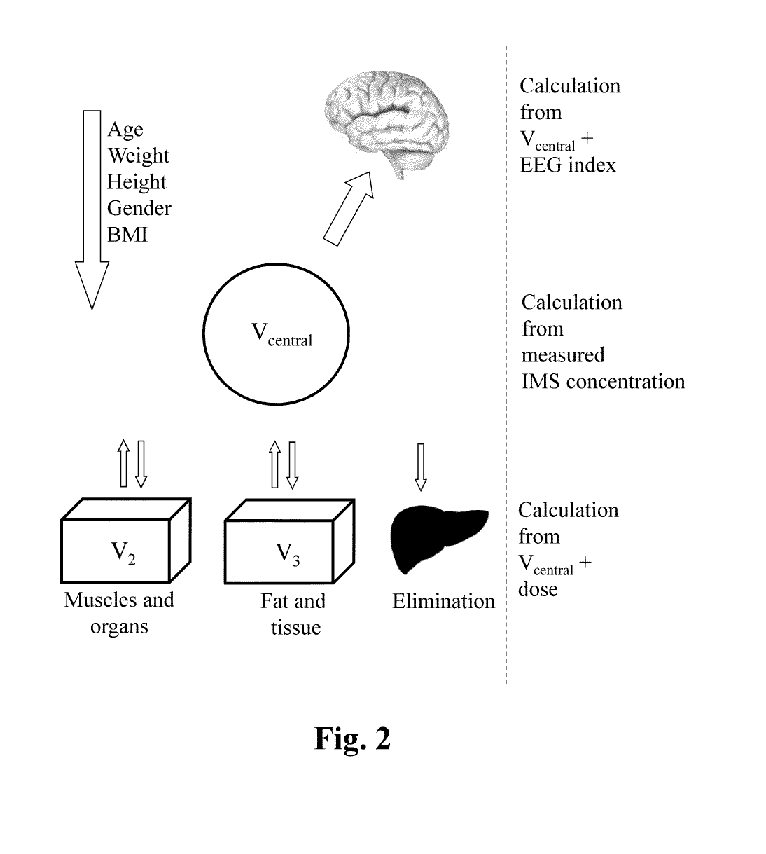

FIG. 2 illustrates schematically the creation of an individualized PK/PD model according to an exemplary embodiment of the invention. The exemplary embodiment represented is based on a conventional three-compartment model. This type of model has so far proved itself to be the best in practice for the description and interpretation of pharmacokinetic processes occurring within the body. In the case of the three-compartment model, the body is divided into a central compartment (V_{central}) and two parallel peripheral compartments (V₂ and V₃). The central compartment V_{central} thereby corresponds to the volume of blood as well as the organs with a high proportion of the cardiac output, in particular the brain, heart and lungs. One of the peripheral compartments (V₂) corresponds to the muscles and other organs, while the other peripheral compartment (V₃) describes the fat and connective tissue. In addition, the elimination of the drug in question takes into account whether the propofol passes essentially via the liver.

The conventional three-compartment models, such as are known from the prior art and are used in clinical practice today have, as inputs, only the demographic data of the patient and generally include the age, the weight, the height, the gender and the BMI. For healthy patients, these models result in an inaccuracy of about 20%. Accordingly, the dose of the anesthetic agent administered is also inaccurate as it is also based on the models calculated for disabled patients.

On the other hand, an individual patient-specific PK/PD model according to an exemplary embodiment of the present invention is calculated during anesthesia not only on the basis of the demographic data of the patient, but also on the concentration levels of the administered anesthetic agent measured in real-time or quasi real-time, while, in addition, the measured EEG index values are integrated into the model calculation. In this way, individualized anesthesia or analgo-sedation control may be performed that offers both a detailed statement about the current state of anesthesia or analgo-sedation and predicts their further development.

Specifically, the size of the central compartment V_{central} is not only determined across-the-board on the basis of the demographic data of the patient, but is individually calculated by the IMS from the actually measured concentrations of administered anesthetic agent. This result, together with the administered dose, flows into the calculation of the exchange process with the peripheral compartments V₂ and V₃ as well as the elimination process. The effect of the administered anesthetic agent is modeled on the basis of the determined size of the central compartment V_{central} in combination with the recorded EEG index values. In the context of an expanded embodiment, the conventional three-compartment model is extended to include additional compartments for calculating the individual patient-specific PK/PD model.

According to one exemplary embodiment of the invention, an anesthesia monitor is implemented that enables the anesthesiologist supplying the anesthetic to develop optimized anesthetic or analgo-sedation control based on an optimized pharmacological model for individual patients. The anesthesia monitor can provide the anesthesiologist with all relevant information. Thus, for example, the above-described networking of an EEG monitor system to measure the effect of the administered anesthesia agent during anesthesia enables the dose-response curve of the patient to be calculated. By means of the integration of the amount supplied and the demographic data of the patient, a prediction about the future course may then be made. In addition, a comparison may be made with pharmacological averages. This allows a statement to be made as to whether the individual patient exhibits a normal, faster or slower metabolism of the administered anesthetic, especially propofol. With a view to the widest possible simplification of anesthesia or analgo-sedation control for the anesthesiologist, the following values/parameters are preferably displayed on the anesthesia monitor:
Measured end-tidal concentration of propofol
Individually calculated propofol blood concentration
Individually calculated propofol effective concentration
Dose-response curve of propofol/EEG index value
Rate of metabolism of propofol

Various correlation analyses may be performed in order to further optimize the PK/PD model based on the actually measured concentrations in the exhaled air of the patient, and to improve its validity. Thus, for example, a correlation analysis between a propofol blood concentration subsequently measured in the laboratory and the end-tidal concentration of propofol measured during the intervention will contribute to more accurate information with respect to the actually existing propofol blood concentration flowing into the model. Correlation analyses between clinical endpoints (e.g. loss of consciousness) and the measured end-tidal concentration of propofol, and correlation analyses between EEG index values and the measured end-tidal concentration of propofol could also contribute to a further improvement.

Regarding further advantageous embodiments of the device according to the invention and to avoid repetition, reference is made to the general part of the description and to the appended patent claims.

Finally, it is expressly pointed out that the above-mentioned exemplary embodiments of the device according to the invention are used only to explain the claimed teaching, but do not restrict the exemplary embodiments.

### LIST OF REFERENCE NUMERALS

1 patient
2 measuring device
3 ion mobility spectrometer
4 means to determine the effect
5 metering device
6 TCI syringe pump
7 data processing device
8 EEG module

1-15. (canceled)

16. Apparatus for performing at least one of anesthesia or analgosedation, said apparatus comprising:
a metering device (5) configured to intravenously administer an adjustable dose of at least one anesthetic agent to a patient (1);
a measuring device (2) configured to determine the concentration of the at least one anesthetic agent in the exhaled air of the patient (1);
means (4) to determine an effect of the at least one anesthetic agent on the patient (1); and
a data processing device (7) configured to communicate via one or more interfaces with the metering device (5),
wherein:
the measuring device (2) and the means (4) are configured to:
determine the effect in order to create an individualized pharmacological model on the basis of the determined values of the parameters for dosage, concentration, and the effect of the at least one anesthetic agent on the patient (1); and
calculate the optimized dosage for the patient (1) of the at least one anesthetic agent on the basis of the created individualized pharmacological model.

17. Apparatus according to claim 16, wherein the metering device (5) comprises a syringe pump (6).

18. Apparatus according to claim 16, wherein the measuring device (2) configured to determine the concentration of the at least one anesthetic agent, works continuously.

19. Apparatus according to claim 16, wherein the measuring device (2) comprises an ion mobility spectrometer (3) with multi-capillary columns.

20. Apparatus according to claim 19, wherein the ion mobility spectrometer (3) is coupled to a flow or CO₂ sensor.

21. Apparatus according to claim 16, wherein the means (4) to determine the effect of the at least one anesthesia agent on the patient (1) comprises a device to derive an EEG-EEG module (8).

22. Apparatus according to claim 16, wherein demographic data of the patient (1) may be entered in the data processing device (7) and included in the creation of the individualized pharmacological model.

23. Apparatus according to claim 16, wherein the at least one anesthetic agent comprises a narcotic.

24. Apparatus according to claim 16, wherein the at least one analgesic agent comprises an opioid.

25. Apparatus according to claim 16, wherein the at least one anesthetic agent comprises a muscle relaxant.

26. Apparatus according to claim 16, wherein the data processing device (7) is configured to calculate the interactions between an anesthetic administered to the patient (1) and an analgesic administered to the patient (1).

27. Apparatus according to claim 16, further comprising a display device on which the calculated optimized dose of the at least one anesthetic agent is represented as a recommendation for the patient (1).

28. Apparatus according to claim 16, wherein the data processing device (7) generates one or more control signals and transmits the one or more control signals to the metering device (5) for automatic adjustment of the calculated optimized dosage of the at least one anesthetic agent for the patient (1).

29. Apparatus according to claim 21, wherein the data processing device (7) is configured to perform a correlation analysis between the EEG index values determined by means of the EEG module (8) and the measured concentration of the at least one anesthetic agent in the air exhaled by the patient (1).

30. A method of operating an apparatus for performing anesthesia, in particular according to claim 16, comprising the steps:
intravenously administering an adjustable dose of the at least one anesthetic agent to a patient (1);
determining a concentration of the at least one anesthetic agent in the exhaled air of the patient (1);
determining an effect of the at least one anesthetic agent on the patient (1);
creating an individualized pharmacological model for the patient (1) based on the parameters for the dosage, concentration and effect of the at least one anesthetic agent or, respectively, the determined values; and
determining an individual optimized dose of the at least one anesthetic agent for the patient (1) on the basis of the created individualized pharmacological model.

31. Apparatus according to claim 16, wherein the effect comprises at least one of an anesthetic depth or an analgosedation depth.

32. Apparatus according to claim 17, wherein the syringe pump (6) is microprocessor-controlled.

33. Apparatus according to claim 16, wherein the measuring device (2) configured to determine the concentration of the at least one anesthetic agent works at measurement intervals of less than 30 s.

34. Apparatus according to claim 33, wherein the measurement intervals are in a range of 15-25 s.

35. Apparatus according to claim 23, wherein the narcotic is propofol.

36. Method according to claim 30, wherein the effect comprises at least one of an anesthetic depth or an analgosedation depth.
